(19) United States
(12) Patent Application Publication
(10) Pub. No.: US 2025/0281133 A1
(43) Pub. Date: Sep. 11, 2025

(54) PANORAMIC X-RAY IMAGE DISPLAY APPARATUS

(71) Applicants: VATECH Co., Ltd., Gyeonggi-do (KR); VATECH EWOO Holdings Co., Ltd., Gyeonggi-do (KR)

(72) Inventor: Yong Joo YANG, Gyeonggi-do (KR)

(73) Assignees: VATECH Co., Ltd., Gyeonggi-do (KR); VATECH EWOO Holdings Co., Ltd., Gyeonggi-do (KR)

(21) Appl. No.: 18/862,516

(22) PCT Filed: May 2, 2023
(86) PCT No.: PCT/KR2023/005990

§ 371 Date: Nov. 1, 2024

(30) Foreign Application Priority Data
May 2, 2022 (KR) 10-2022-0054342

(51) Int. Cl. A61B 6/46

(52) U.S. Cl. CPC A61B 6/463

(57) ABSTRACT

Proposed is a panoramic X-ray image display apparatus, which subdivides a partial image display area displayed in a part of a background image area and displays a plurality of panoramic images different from each other in the subdivided respective areas, the apparatus including a viewer module configured to display a first panoramic image in the background image area of a screen, display the partial image display area including n (where n is an integer greater than or equal to 2) divided areas in a part of the background image area according to a user command, and display some parts of a plurality of second panoramic images different from each other in the n divided respective areas.

### TECHNICAL FIELD

The present disclosure relates to a radiography apparatus and, more particularly, to a panoramic X-ray image display apparatus configured to display a partial image display area subdivided into a plurality of areas in a part of a background image area where a first panoramic image of a subject is displayed, and display some parts of second panoramic images different from each other for the same subject in the subdivided respective areas according to a user's selection.

The embodiments of the present disclosure are derived from research conducted as part of the support for the Industrial Technology International Cooperation Project of the Korea Institute for Advancement of Technology of the Ministry of Trade, Industry and Energy [Project Number: P0019248, Research Project Name: Development and Commercialization of Advanced X-ray Diagnostic System Specialized in Root Canal Treatment and Early Lesion Diagnosis].

### BACKGROUND ART

In the medical field, an X-ray radiography apparatus is an apparatus configured to emit a predetermined amount of X-rays to a patient's body part to be imaged, detect the X-rays having passed through the body part with an X-ray sensor, and compose X-ray images on the basis of detected electrical signals. The X-rays are transmitted while being attenuated at attenuation rates different depending on materials present along travelling paths thereof, and when reaching the X-ray sensor, the X-rays are converted into the electrical signals by the photoelectric effect. As such, the X-ray radiography apparatus provides information about the inside of a radiography target in the form of X-ray images by using the electrical signals representing the accumulated amount of attenuation along the X-ray travelling paths.

Meanwhile, the capturing of X-ray panoramic images has been widely used in the field of dentistry for a long time, so X-ray panoramic images that are now recognized as essential standard images for diagnosis are obtained and provided. In the capturing of X-ray panoramic images, frame data, i.e., a plurality of X-ray image data obtained by taking pictures of a patient's dental arch for each section, is overlaid and arranged by using a so called shift and add method that is a tomography technique, whereby a focus area is placed on an arbitrary image layer and two-dimensional shape of the dental arch is displayed by spreading it out on a flat surface.

In this case, when the degree of overlay of the frame data is controlled, so-called multilayer panoramic images with positions different from each other of image layers that are focus areas with respect to an X-ray emission direction may be obtained. In particular, when referring to “Panoramic X-ray Imaging Apparatus and Method thereof (Registration No. 10-2079576)” previously filed and registered by the applicant of the present application, not only a plurality of panoramic images is generated with frame data so that a first panoramic image as one of the plurality of panoramic images is displayed in a background image area of a screen, but also a partial image display area is displayed in a part of the background image area so that a part corresponding to a second panoramic image as one of the plurality of panoramic images is displayed in the partial image display area, thereby providing an effect that the depth resolution of the panoramic images is improved and the display efficiency of the panoramic images is improved.

However, such a patented embodiment also has limitations in displayed information because the second panoramic image displayed in the partial image display area is limited to one of the plurality of panoramic images.

### DocumENTS OF RELATED ART

### Patent Documents

(Patent Document 1) Korean Patent No. 10-2079576
(Patent Document 2) Korean Patent No. 10-1094180
(Patent Document 3) Korean Patent No. 10-1389841

### DISCLOSURE

### Technical Problem

Accordingly, the embodiment of the present disclosure is devised and created to solve the above-described problem, and a main objective of the present disclosure is to provide a panoramic X-ray image display apparatus capable of displaying partial images of a plurality of second panoramic images different from each other on a part of a background image area where a first panoramic image is displayed.

Furthermore, another objective of the present disclosure is to provide a panoramic X-ray image display apparatus, wherein a partial image display area is displayed in a part of a background image area where a first panoramic image as one of a plurality of panoramic images is displayed, and a user may selectively subdivide the partial image display area and display some corresponding parts of a plurality of second panoramic images different from each other in the subdivided respective areas.

### Technical Solution

According to an exemplary embodiment of the present disclosure to achieve the above-mentioned objectives, there is provided a panoramic X-ray image display apparatus, including:
a storage for storing a plurality of X-ray frame data for a subject;
an input unit for user operation;
an image processor for generating a plurality of panoramic images for image layers different from each other by using at least some parts of the X-ray frame data;
a display for providing a screen; and
a viewer module configured to display a background image area on the screen, display a first panoramic image as one of the plurality of panoramic images in the background image area, display a partial image display area including n (where n is an integer greater than or equal to 2) divided areas in a part of the background image area by the user operation, and display some parts of second panoramic images different from each other among the plurality of panoramic images in the n divided respective areas,
wherein the some parts of the second panoramic images respectively displayed in the n divided areas may correspond to the first panoramic image with locations where the n areas are respectively overlaid.

In this case, the viewer module may divide the partial image display area into the n areas according to a user command input through the input unit.

The plurality of panoramic images may differ from each other in at least one of quantity, position, shape, angle, and thickness of the image layers.

Furthermore, the first and second panoramic images may be panoramic images for dental arch trajectories different from each other, and the second panoramic images respectively displayed in the n divided areas may be multilayer panoramic images for the corresponding dental arch trajectories.

In addition, the viewer module may change at least one of alignment relationship and quantity of the n divided areas according to a user operation command through the input unit.

### Advantageous Effects

According to the technical problem solution described above, the panoramic X-ray image display apparatus according to the exemplary embodiment of the present disclosure has an effect that a partial image display area is displayed in a part of a background image area where a first panoramic image as one of a plurality of panoramic images is displayed, and a user may selectively subdivide the partial image display area and display some corresponding parts of a plurality of second panoramic images different from each other in the subdivided respective areas, thereby providing partial images from various positions and angles.

Furthermore, the panoramic X-ray image display apparatus according to the exemplary embodiment of the present disclosure has another effect that the display efficiency of panoramic images is improved and detailed information within a dental arch desired by a user is provided as various images through subdivided partial image display areas, thereby realizing precise and accurate diagnosis in the medical field.

### DESCRIPTION OF DRAWINGS

FIG. 1 is an exemplified view illustrating a configuration of a panoramic X-ray image display apparatus according to an exemplary embodiment of the present disclosure.

FIG. 2 is an exemplified view illustrating a scan sequence performed by the panoramic X-ray image display apparatus for describing the exemplary embodiment of the present disclosure.

FIG. 3 is a view illustrating processes of obtaining a plurality of frame data and reconstructing panoramic images by using the same according to the performing of the scan sequence shown in FIG. 2.

FIG. 4 is an exemplified view illustrating a display screen displayed by the panoramic X-ray image display apparatus.

FIG. 5 is an exemplified view illustrating an alignment relationship between a first panoramic image and second panoramic images.

FIG. 6 is an exemplified flowchart illustrating a panoramic image processing of the panoramic X-ray image display apparatus according to the exemplary embodiment of the present disclosure.

FIG. 7 is an enlarged view illustrating a partial image display area according to the exemplary embodiment of the present disclosure.

### BEST MODE

Hereinafter, various exemplary embodiments of the present disclosure will be described with reference to the drawings. The technical idea of the present disclosure may be more clearly understood through the exemplary embodiments. In addition, the present disclosure is not limited to the exemplary embodiments described below, but may be modified in various forms within the scope of the technical idea to which the present disclosure belongs. Meanwhile, since the same drawing numeral indicates a component having the same characteristics, a description of a component having the same drawing numeral as that mentioned in the description of one drawing may be omitted in the description of another drawing.

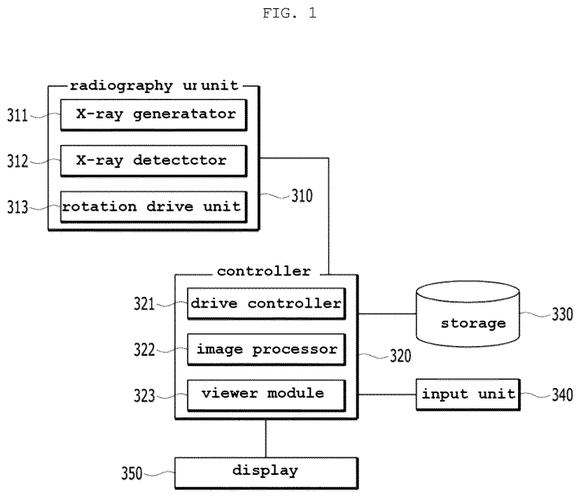

FIG. 1 is a view illustrating a configuration of a panoramic X-ray image display apparatus according to an exemplary embodiment of the present disclosure.

The panoramic X-ray image display apparatus according to the exemplary embodiment of the present disclosure includes: a radiography unit 310 including an X-ray generator 311 and an X-ray detector 312; a controller 320 including an image processor 322 and a viewer module 323; a storage 330 for storing X-ray frame data, image data, and panoramic image data; an input unit 340 for performing a user interface function; and a display 350 for displaying an image.

For reference, the radiography unit 310 is provided with a rotation drive unit 313 for rotating and/or moving the X-ray generator 311 and the X-ray detector 312 in directions opposite to each other, and the controller 320 is provided with a drive controller 321 for controlling the operation of the rotation drive unit 313, the X-ray generator 311, and the X-ray detector 312.

The controller 320 may be configured to control all actions of the panoramic X-ray image display apparatus linked to not only the image processor 322 and the viewer module 323 but also the storage 330, input unit 340, and display 350, in addition to the central processing unit. The radiography unit 310 may be connected to the controller 320 by wire or wirelessly.

The radiography unit 310 includes the X-ray generator 311 and the X-ray detector 312, which rotate and/or move in directions opposite to each other with respect to a patient's dental arch placed therebetween by the rotation drive unit 313. In this case, a rotation axis therefor may also move one-dimensionally or two-dimensionally. When a scan sequence for obtaining X-ray panoramic images starts, the X-ray generator 311 emits X-rays toward a subject in parallel with the aforementioned rotation and/or movement operations, and the X-ray detector 312 receives the X-rays passing through the subject in frame units so as to obtain a plurality of X-ray frame data. Hereinafter, the X-ray image data in a plurality of frames formed by X-ray beams that are emitted from various positions and angles during performing a series of scan sequences and then reach the X-ray detector 312 will be referred to as a plurality of frame data, and such frame data is also stored in the storage 330.

The controller 320 stores the plurality of frame data obtained from the radiography unit 310 in the storage 330, and the image processor 322 extracts the plurality of frame data stored in the storage 330 and reconstructs multilayer panoramic images for a plurality of image layers. The plurality of panoramic images reconstructed in this manner may be displayed on a screen by the viewer module 323.

The viewer module 323 also capable of being included as part of the image processor 322 may be implemented with firmware/software modules having a series of functions to display a viewer screen in a predetermined format on the display 350 according to a previously stored algorithm, appropriately display the X-ray panoramic images reconstructed by the image processor 322 according to a user's command input through the input unit 340, and provide additional functions required by the user.

For example, the viewer module 323 may display a background image area on a screen, display a first panoramic image as one of a plurality of panoramic images in the background image area, display a partial image display area in a part of the background image area according to a user command, divide the partial image display area into n areas (where n is an integer greater than or equal to 2) according to a user command, and respectively display some parts of second panoramic images different from each other among the plurality of panoramic images in the n divided areas according to a user command. In this case, some parts of the second panoramic images displayed in the n divided respective areas correspond to the first panoramic image with positions where the n divided respective areas are overlaid. This will be discussed below. For reference, the reconstructed X-ray panoramic images may be stored again in the storage 330.

In the panoramic X-ray image display apparatus including the above-described components, the controller 320, storage 330, input unit 340, and display 350 may be implemented in such forms of a single or a plurality of computer devices and peripheral devices thereof.

The input unit 340 may be a mouse. In addition, the input unit 340 may include a keyboard, keypad, touchpad, and the like of a computer, and the type of input means is not limited thereto. For example, the input unit 340 is controllable by using the input means exemplified, and may also include a graphic user interface (GUI) displayed on the display 350 via the controller 320.

As described above, the controller 320 is configured to include a central processing unit (CPU) for generally controlling the operation of the panoramic X-ray image display apparatus according to the present disclosure. As an example, the controller 320 may be implemented by using at least one of application specific integrated circuits (ASICs), digital signal processors (DSPs), digital signal processing devices (DSPDs), programmable logic devices (PLDs), field-programmable gate arrays (FPGAs), processors, controllers, micro-controllers, and microprocessors. The controller 320 may also be implemented with the firmware/software modules executable on the aforementioned hardware platform. In this case, the firmware/software modules may be implemented with one or more software applications developed in a suitable program language.

The storage 330 is a digital data storage medium, and performs a function of storing not only X-ray frame data obtained from the X-ray detector 312, but also data related to settings of various images, parameter values, and the like for performing the operation of a panoramic X-ray image processing apparatus, in addition to X-ray image data, panoramic images, and the like, generated during an image processing process or reconstructed as a result of the image processing.

The display 350 is an image display device capable of outputting X-ray panoramic images and a viewer screen configured in a predetermined format.

The panoramic X-ray image display apparatus according to the exemplary embodiment of the present disclosure including the above-described components may at least display a first panoramic image as one of a plurality of panoramic images in a background image area of a screen, and display some parts of second panoramic images, which are different from each other among the plurality of panoramic images, in the n divided respective areas according to a user's selection. In this case, some parts of the second panoramic images displayed in the n divided respective areas correspond to the first panoramic image with the positions where the n divided respective areas are overlaid.

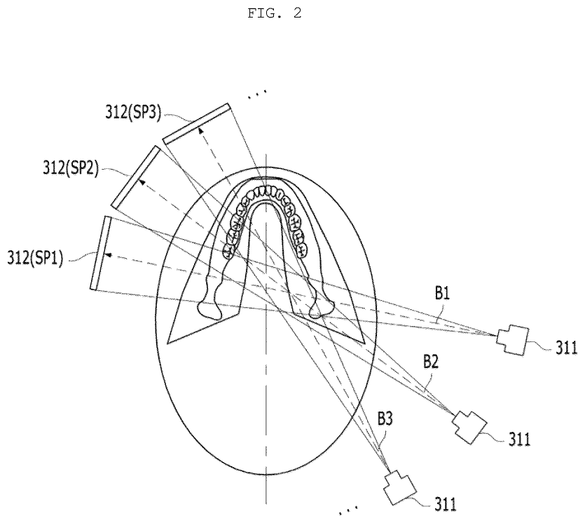

FIG. 2 is an exemplified view illustrating a scan sequence performed by the panoramic X-ray image display apparatus for describing the exemplary embodiment of the present disclosure.

Referring to FIG. 2, an X-ray generator 311 and an X-ray detector 312 rotate and/or move in directions opposite to each other with respect to a patient's dental arch placed therebetween. In such a process, the X-ray detector 312 receives each of X-ray beams B1, B2, B3, . . . , and so on, passing through various locations within an dental arch at various angles, and generates X-ray frame data including X-ray reception signals of one frame for each location SP1, SP2, SP3, . . . , and so on. One scan sequence is performed in such a manner. The scan sequence refers to a series of processes in which a radiography unit 310 moves continuously along a predetermined movement trajectory and obtains a plurality of frame data, which is a plurality of X-ray image data obtained in frame units from the X-ray detector 312. A technique for obtaining data required for reconstructing X-ray panoramic images of a plurality of image layers different from each other through one scan sequence is proposed in the applicant's previous patent document, Korean Patent No. 10-0917679.

Here, one scan sequence may also be configured to include movements of the radiography unit 310, the movements being identical or similar to conventional panoramic image capturing operations.

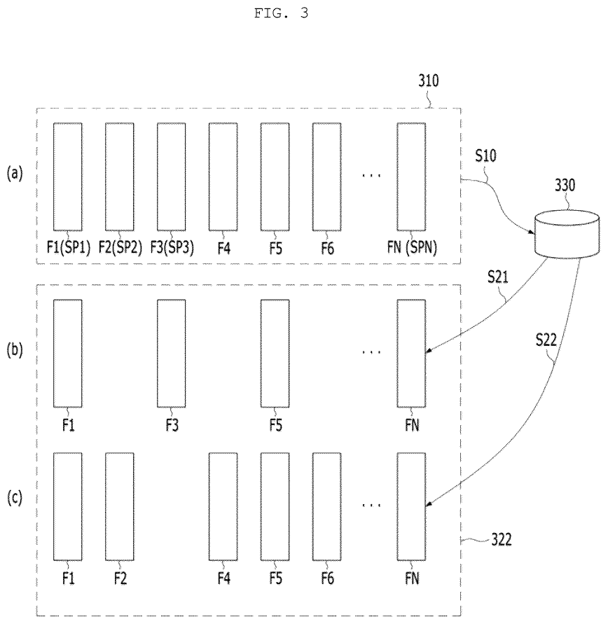

FIG. 3 is a view illustrating processes of obtaining a plurality of frame data and reconstructing panoramic images by using this frame data according to the performing of the scan sequence shown in FIG. 2.

View (a) illustrates a plurality of frame data F1, F2, F3, F4, F5, F6, ˜, FN obtained in step S10 through one scan sequence in the radiography unit 310. As the same as first frame data F1 generated as a result of receiving a first X-ray beam B1 at a location SP1, second frame data F2 generated as a result of receiving a second X-ray beam B2 at a location SP2, third frame data F3 generated as a result of receiving a third X-ray beam B3 at a location SP3, and so on, which are generated by an X-ray detector 312 (see FIG. 2), the plurality of frame data F1˜FN is composed of the X-ray image data in a plurality of frame units and obtained by receiving X-ray beams passing through a portion of an dental arch, which is a radiography target area, at various angles at a plurality of viewpoints through the X-ray detector 312 while the radiography unit 310 performs one scan sequence. Such a plurality of frame data F1, ˜, FN is stored in the storage 330 described above. In this case, each of the first frame data F1, ˜, FN may store information about the positions and directions of the X-ray beams penetrating within the radiography target area.

Views (b) and (c) schematically illustrate processes of extracting, from the storage 330, a plurality of frame data required to reconstruct respective first and second X-ray panoramic images in the aforementioned image processor 322. More specifically, view (b) illustrates frame data F1, F3, F5, . . . , FN of a first group and extracted from a population of the plurality of frame data in order to perform step S21 of reconstructing the first X-ray panoramic image. View (c) illustrates frame data F1, F2, F4, F5, F6, . . . , FN of a second group and extracted from a population of the plurality of frame data in order to perform step S22 of reconstructing the second X-ray panoramic images different from the first X-ray panoramic image.

However, among the plurality of frame data, the frame data of the first group and the frame data of the second group satisfy the following conditions. The composition of the frame data forming the first group and the composition of the frame data forming the second group may be the same to or different from each other. The number of frames of the frame data constituting the first group and the number of frames of the frame data constituting the second group may be the same to or different from each other.

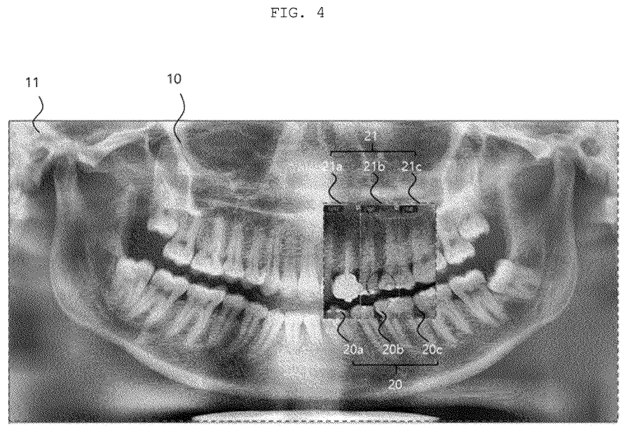

FIG. 4 is an exemplified view illustrating a display screen displayed by the panoramic X-ray image display apparatus.

On a screen output through the panoramic X-ray image display apparatus according to the exemplary embodiment of the present disclosure, not only there is provided a background image area 11 for displaying a first panoramic image 10, which is an X-ray panoramic image for at least one of first image layers, but also there is provided, as an area disposed in a predetermined part within the background image area 11 and corresponding to the predetermined part, a partial image display area 21 for displaying some parts of second panoramic images 20, which are X-ray panoramic images of second image layers that are at least partially different from the first image layer.

One or more of the partial image display areas 21 may be provided according to a user command, and each partial image display area 21 may be divided into n areas (where n is greater than or equal to 2, or is in the form of a*b). In addition, some parts of the second panoramic images 20a, 20b, and 20c for second image layers different from each other may respectively be displayed in the n divided areas 21a, 21b, and 21c according to a user's selection.

The n divided areas 21a, 21b, and 21c are for providing the user with more information about a dental arch structure of a subject by respectively displaying some parts of the second panoramic images 20a, 20b, and 20c thereon, which are different from the first panoramic image 10, according to the user's selection, so shapes thereof may vary. For example, as for the n divided areas 21a, 21b, and 21c, it is possible to divide the partial image display area 21 into two or more vertical areas as shown in the drawing, or differently, it is also possible to divide the partial image display area 21 into two or more horizontal areas or horizontal and vertical areas in the form of an a*b matrix. In addition, an alignment relationship of the n divided areas 21a, 21b, and 21c may be selected by the user. Alternatively, the size, position, shape, quantity, or the like of the partial image display area 21 may also be adjusted according to the user's command.

In a case where the user first checks the first panoramic image 10 provided through the background image area 11 and determines that additional review of a certain part is required, the partial image display area 21 may be selectively activated according to the user's command, so as to replace the first panoramic image of the certain part, whereby corresponding parts of the second panoramic images 20a, 20b, and 20c different from each other may be displayed in the n divided respective areas 21a, 21b, and 21c. The second panoramic images 20 may also be displayed with different brightness or color compared to that of the first panoramic image 10.

To this end, the image processor 322 reconstructs not only the first panoramic image 10 for at least one first image layer from X-ray image data in a plurality of frames obtained by the radiography unit 310 through one scan sequence, but also the second panoramic images 20 for a plurality of second image layers that are at least partially different from the first image layer, thereby providing the reconstructed images to the viewer module 323.

For reference, the first panoramic image is a panoramic image for at least one first image layer, and the plurality of respective second panoramic images are panoramic images for the plurality of second image layers, which are different from each other and are at least partially different from the first image layer. In a case where the first panoramic image is a panoramic image for any one image layer, the second panoramic images may be panoramic images for the plurality of image layers that differ from the first image layer of the first panoramic image.

In addition, the second panoramic images may be panoramic images of the plurality of image layers for a predetermined dental arch shape. In the case of a typical panoramic X-ray image display apparatus, various types of dental arch trajectories including types of normal, wide, narrow, and child are stored in advance according to a dental arch shape for each subject, and panoramic images for a dental arch trajectory selected for each subject is provided.

The panoramic image display apparatus according to the exemplary embodiment of the present disclosure displays a first panoramic image 10 for a first image layer in a background image display area 11, and displays corresponding some parts of second panoramic images 20a, 20b, and 20c for second image layers different from each other in n divided respective areas 21a, 21b, and 21c of a partial image display area 21. In this case, the first and second image layers may be image layers respectively corresponding to dental arch trajectories different from each other among preset dental arch trajectories, and a user may check the first panoramic image 10 for the first image layer of an arbitrary first dental arch trajectory (for example, “normal”) through the background image display area 11, and check the second panoramic images 20a, 20b, and 20c for the plurality of second image layers, which are respective multilayer panoramic images of second dental arch trajectories (for example, “wide”) different from the first dental arch trajectory through the divided areas of the partial image display area 21.

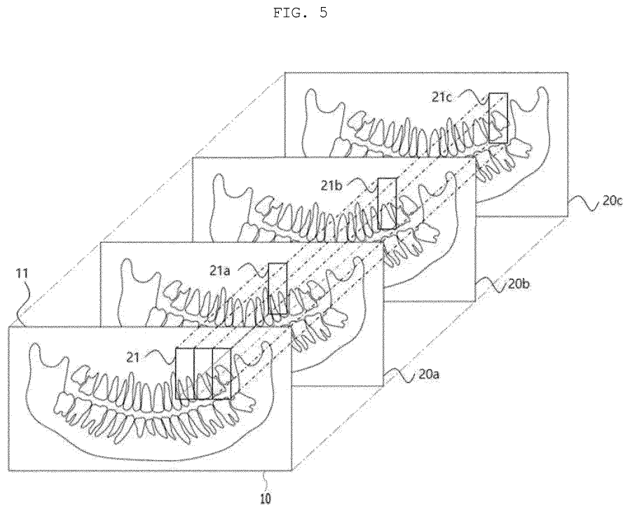

FIG. 5 is an exemplified view illustrating an alignment relationship between a first panoramic image and second panoramic images.

Assuming that a partial image display area 21 is divided into three horizontal areas as the same as described above, second panoramic images 20a, 20b, and 20c are stored as panoramic images having the same frame size and the same magnification ratio and being represented with the same magnification and the same range in plane as those of a first panoramic image 10. However, in a display screen illustrated in FIG. 4, only parts of second panoramic images 20a, 20b, and 20c respectively corresponding to the n divided areas 21a, 21b, and 21c of a partial image display area 21 are displayed on the screen. As described above, in the displaying method, the n divided areas 21a, 21b, and 21c of the partial image display area 21 in the plurality of second panoramic images 20a, 20b, and 20c may be displayed to replace or overlap the parts corresponding to the n divided areas 21a, 21b, and 21c in the first panoramic image 10.

In addition, for example, the user may divide the partial image display area 21 into the n divided areas 21a, 21b, and 21c by operating an input unit while the partial image display area 21 is activated, and select the second image layers of the second panoramic images displayed in the n divided areas 21a, 21b, and 21c through the operation of the input unit, such as turning a mouse wheel in a forward or backward direction in each of the n divided areas 21a, 21b, and 21c.

As described above, the panoramic X-ray image display apparatus according to the exemplary embodiment of the present disclosure displays, through a display screen, a first panoramic image for at least one first image layer and some parts of second panoramic images for a plurality of second image layers, which are at least partially different from the first image layer.

Alternatively, the panoramic X-ray image display apparatus may display, as a background image, the first panoramic image for the first image layer, arrange n divided partial image display areas on a part of the background image, and show some parts of second panoramic images for second image layers different from each other on the n divided respective areas.

Hereinafter, from a process of obtaining X-ray image data by a panoramic image display apparatus to a process of displaying first and second panoramic images, specific examples are described therewith.

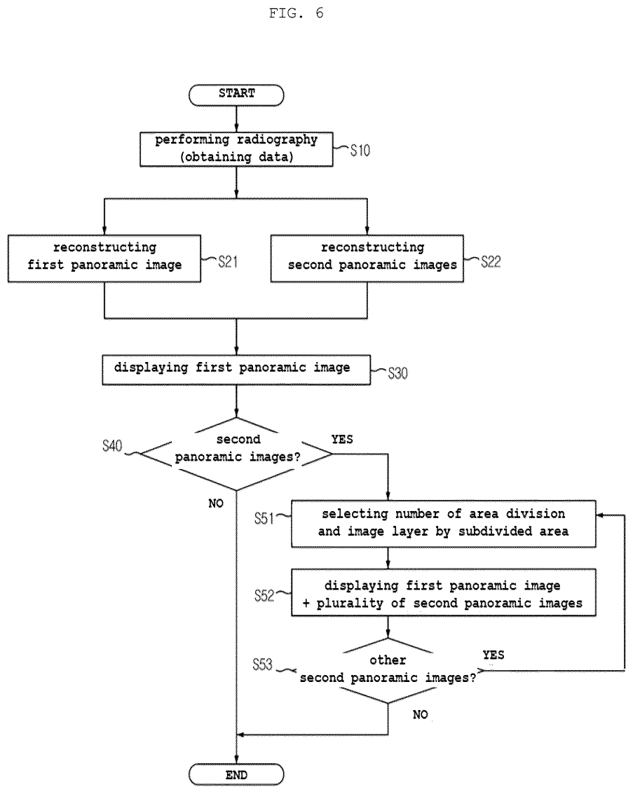

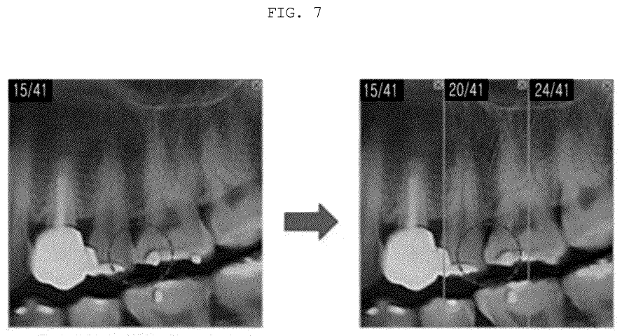

First, FIG. 6 is an exemplified flowchart illustrating a panoramic image processing of the panoramic X-ray image display apparatus according to the exemplary embodiment of the present disclosure, and FIG. 7 is an enlarged view illustrating a partial image display area according to the exemplary embodiment of the present disclosure.

First, in step s10, X-ray radiography is performed by a radiography unit 310. This involves preset movements of the radiography unit 310 including an X-ray generator 311 and an X-ray detector 312. The movements of the radiography unit 310 may be configured to include a series of continuous movements in which the X-ray generator 311 and the X-ray detector 312 rotate and/or move in a state of facing each other while having a subject disposed therebetween. The operation of the X-ray generator 311 and X-ray detector 312 synchronized with such movements constitutes a single scan sequence. X-ray image data of a plurality of frames obtained through the scan sequence is stored in a storage. The X-ray image data of the plurality of frames may include information such as locations and directions of X-ray beams passing through the subject and forming each frame.

Next, an image processor 322 performs step s21 of reconstructing a first panoramic image, and performs step s22 of reconstructing a plurality of second panoramic images different from each other. The plurality of second panoramic images different from each other may be defined as second panoramic images for image layers different from each other. The first panoramic image as well as the plurality of second panoramic images different from each other, which are reconstructed in the image processor 322, are also stored in a storage 330.

Meanwhile, a viewer module 323 performs step S30 of displaying the first panoramic image on a panoramic image display part, i.e., a background image area 11. In such a case, in step s40, it is determined whether or not a user requests, through an input unit, display of the plurality of second panoramic images in a partial area of the first panoramic image, i.e., a partial image display area 21.

When there is a request for displaying the plurality of second panoramic images in step s40, the viewer module 323 requests an input of a dividing method and the number thereof for the partial image display area 21 and selection of an image layer for each of the n divided areas. Naturally, the number of divided areas for the partial image display area 21 may be predetermined, or the user may also be allowed to select the number within a predetermined range. In addition, the second panoramic images (also called as subdivided images) respectively displayed in the n divided areas may also be determined as panoramic images for predetermined image layers. In addition, the second panoramic images may be determined as multilayer panoramic images corresponding to a dental arch shape (i.e., normal, narrow, wide, etc.) selected by the user.

When the user inputs the dividing method and the number thereof for the partial image display area 21 and the selection of the image layer for each of the n divided areas, the viewer module 323 subdivides the partial image display area 21 by using the dividing method and the number of divided areas, which are selected by the user, and performs step s52 of displaying some parts of the second panoramic images corresponding to the selected image layers in the n divided areas. In this case, the first panoramic image is displayed in the background image area 11, and the partial image display area 21 is located in a partial area of the first panoramic image. Some parts of the second panoramic images displayed in the n divided areas within the partial image display area 21 correspond to the first panoramic image with positions where the n divided (subdivided) respective areas are overlaid. That is, each of the second panoramic images displayed as a divided image corresponds to a predetermined portion of the first panoramic image, and are overlaid or replaced on the predetermined portion of the first panoramic image.

For reference, FIG. 7 illustrates that second panoramic images respectively corresponding to three image layers are displayed in a partial image display area 21 divided into three by a user, and the left view thereof illustrates the partial image display area 21 before segmentation and the right view thereof illustrates the partial image display area 21 after being divided into the three areas. In respective areas of the partial image display area 21 divided into three, some parts of the second panoramic images corresponding to 15-th/20-th/24-th image layers are displayed, respectively. In particular, when a part within a red circle is examined, it is difficult to distinguish a gap between two teeth with a second panoramic image of the partial image display area before segmentation as shown in the left view, but as shown in the right view, a gap between the two teeth may be clearly recognized with a second panoramic image of the other image layer after segmentation.

Therefore, the user may subdivide the partial image display area 21 into two or more as required, and display one subdivided image among the plurality of panoramic images in each subdivided area, thereby being able to observe more information.

Accordingly, the panoramic X-ray image display apparatus according to the exemplary embodiment of the present disclosure has the effect that the display efficiency of the panoramic images are improved and detailed information within a dental arch desired by a user is displayed and provided as various images through the subdivided partial image display areas.

Meanwhile, while displaying the plurality of second panoramic images through the partial image display area 21 together with the first panoramic image, the user may select the positions, angles, quantities, thicknesses, or the like of the image layers to be displayed with the second panoramic images displayed in the partial image display area 21, and is enabled to select removal or position movement of the partial image display area 21 as well.

Furthermore, the viewer module 323 may additionally display an indicator for indicating relative positional relationships of the second panoramic images overlaid on the first panoramic image.

The present disclosure has been described with reference to the exemplary embodiments shown in the drawings, but these are only exemplary, and those skilled in the art will appreciate that various modifications and other equivalent embodiments are possible. Therefore, the true technical protection scope of the present disclosure should be determined by the technical idea of the appended claims.

1. A panoramic X-ray image display apparatus, comprising:
a storage for storing a plurality of X-ray frame data for a subject;
an input unit for user operation;
an image processor for generating a plurality of panoramic images for image layers different from each other by using at least some parts of the X-ray frame data;
a display for providing a screen; and
a viewer module configured to display a background image area on the screen, display a first panoramic image as one of the plurality of panoramic images in the background image area, display a partial image display area comprising n (where n is an integer greater than or equal to 2) divided areas in a part of the background image area by the user operation, and display some parts of second panoramic images different from each other among the plurality of panoramic images in the n divided respective areas,
wherein the some parts of the second panoramic images respectively displayed in the n divided areas correspond to the first panoramic image with locations where the n areas are respectively overlaid,
wherein the first and second panoramic images are panoramic images for dental arch trajectories different from each other, and the second panoramic images respectively displayed in the n divided areas are multilayer panoramic images for the corresponding dental arch trajectories.

2. The panoramic X-ray image display apparatus of claim 1, wherein the viewer module divides the partial image display area into the n areas according to a user command input through the input unit.

3. The panoramic X-ray image display apparatus of claim 1, wherein the plurality of panoramic images differ from each other in at least one of quantity, position, shape, angle, and thickness of the image layers.

4. (canceled)

5. The panoramic X-ray image display apparatus of claim 1, wherein the viewer module changes at least one of alignment relationship and quantity of the n divided areas according to a user operation command through the input unit.
